(19) United States
(12) Patent Application Publication
(10) Pub. No.: US 2018/0147238 A1
(43) Pub. Date: May 31, 2018

(54) COMPOSITION OF IRON SUCROSE AND PROCESS FOR ITS PREPARATION

(71) Applicant: SUN PHARMACEUTICAL INDUSTRIES LTD., Mumbai (IN)

(72) Inventors: Rajamannar THENNATI, Baroda (IN); Shriprakash Dhar DWIVEDI, Baroda (IN); V S N Murty KADIYALA, V, Baroda (IN); Kanaksinh Jesingbhai JADAV, Baroda (IN); Bharat Avcharbhai KAILA, Baroda (IN); Keyur Shashikant THAKER, Baroda (IN); Binaka Rahul DOLE, Baroda (IN)

(73) Assignee: SUN PHARMACEUTICAL INDUSTRIES LTD., Mumbai (IN)

(21) Appl. No.: 15/577,489

(22) PCT Filed: May 27, 2016
(86) PCT No.: PCT/IN2016/050158

§ 371 Date: Nov. 28, 2017

(30) Foreign Application Priority Data
May 29, 2015 (IN) 2110/MUM/2015

(51) Int. Cl. A61K 33/26; A61P 7/06

(52) U.S. Cl. CPC A61K 33/26; A61P 7/06

(57) ABSTRACT

The present invention relates to composition of iron sucrose and process for its preparation. The present invention also related to a process for the preparation of iron sucrose suitable for the iron sucrose composition.

### RELATED APPLICATIONS

This application claims the benefit of Indian Patent Application no. 2110/MUM/2015 filed on May 29, 2015 which is hereby incorporated by reference.

### FIELD OF INVENTION

The present invention relates to compositions of iron sucrose and processes for its preparation. The present invention also relates to a process for the preparation of iron sucrose suitable for the iron sucrose composition.

### BACKGROUND OF INVENTION

Iron sucrose is complex of polynuclear iron (III)-hydroxide in sucrose which was approved by US FDA in the year 2000 for the treatment of iron deficiency anemia in patients with chronic kidney disease (CKD). Iron sucrose injection has a molecular weight of approximately 34,000 to 60,000 daltons and a proposed structural formula:

[Na₂Fe₅O₈(OH)·3(H₂O)]n·m(C₁₂H₂₂O₁₁)

where: n is the degree of iron polymerization and m is the number of sucrose molecules associated with the iron (III)-hydroxide. Luitpold is marketing Iron sucrose under the brand name Venofer® in US and other markets across the globe.

There are many references on processes for preparation of Iron Sucrose. U.S. Pat. No. 7,964,568 discloses a process for the preparation of a high molecular weight iron-saccharidic complex suitable for parenteral administration in human or veterinary medicine. The process disclosed therein involves preparation of an aqueous solution, aqueous colloid, hydrosol or mixtures thereof comprising Fe(III) ion in the presence of (OH)⁻ ion produced in situ from a ferric salt that is substantially fully reacted with a basic reactant and further comprising the addition, to the reaction mixture prior to the iron-saccharidic complex achieving its final molecular weight, of at least one sugar or sugar derivative molecular weight moderating additive.

Following references also disclose processes for the preparation of Iron Sucrose: U.S. Pat. No. 7,674,780, U.S. Pat. No. 8,053,470, US20080167266, WO 2009078037, U.S. Pat. No. 8,030,480 and U.S. Pat. No. 8,053,470.

The present inventors have found a process for the preparation of iron sucrose which provides predictable and reproducible control of the molecular weight range of iron sucrose obtained by the process. The present inventors surprisingly found that compositions prepared using the above said iron sucrose were such that the molecular weight of the iron sucrose was substantially unaltered when the composition is subjected to steam sterilization.

### SUMMARY

The present invention also provides a composition comprising iron sucrose dissolved in an aqueous vehicle wherein the iron sucrose is characterized by a molecular weight in the range 45000 to 60000 Dalton and wherein the molecular weight of iron sucrose is substantially unaltered when the composition is filled in a suitable container and is subjected to steam sterilization.

Advantageously, compositions prepared using the iron sucrose prepared by the processes disclosed herein were such that the molecular weight of the iron sucrose was substantially unaltered when the composition is subjected to steam sterilization.

The composition of the present invention can be prepared by a process comprising:
a) gradually adding an aqueous sodium carbonate solution over a period of 20 to 80 minutes to an aqueous ferric chloride solution at temperature of 10° C. to 30° C. wherein, the quantity of sodium carbonate is 1.2 to 1.8 moles per mole of ferric chloride and isolating ferric oxyhydroxide cake thus formed,
b) suspending the ferric oxyhydroxide cake in water and adding sucrose to obtain a reaction mass wherein quantity of sucrose is 4 to 8 moles per mole of ferric chloride in step a,
c) adding sodium hydroxide to the reaction mass to obtain a pH of 10-12 and heating it to temperature of 100° C. to 106° C. for 12-36 hours,
d) adding methanol and filtering the reaction mass to obtain wet cake
e) preparing sodium sucrosate solution by dissolving sucrose and sodium hydroxide in a aqueous parenteral vehicle and heating at a temperature of 99° C. to 103° C. and
f) dissolving the wet cake in sodium sucrosate solution and removing methanol from the solution.
   and further wherein the volume is adjusted with the parenteral aqueous vehicle and the composition obtained therefrom filled into suitable containers and subjected to steam sterilization.

### Definition

“Molecular weight” of iron sucrose as discussed herein refers to weight average molecular weight (Mw) as determined by the process provided under Iron Sucrose injection monograph in United States Pharmacopoeia—38.

The term “Steam sterilization” refers to a process of sterilization by heating at temperature of about 110° C. to 121° C. for about 10 to 20 minutes under pressure of about 5 to 15 psi.

The phrase “the molecular weight of iron sucrose is substantially unaltered” means that the change in weight average molecular weight of the iron sucrose is not more than ±1000 Daltons.

The term “aqueous vehicle” refers to a solution of sodium hydroxide in water having a pH of 8.0 to 12.0.

The term “aqueous vehicle suitable for parenteral administration” refers to an aqueous vehicle which is rendered suitable for parenteral administration by subjecting it to steam sterilization.

### DESCRIPTION

In one aspect the present invention provides a composition comprising iron sucrose dissolved in aqueous vehicle suitable for parenteral administration wherein the iron sucrose is characterized by a molecular weight in the range 45000 to 60000 Dalton and wherein the molecular weight of iron sucrose is substantially unaltered when the composition is filled in a suitable container and is subjected to steam sterilization.

As an embodiment the molecular weight of the iron sucrose in the composition of the present invention is in the range of 50000 Dalton to 55000 Dalton.

As an embodiment, the present invention also provides a composition comprising iron sucrose dissolved in aqueous vehicle suitable for parenteral administration wherein the iron sucrose is characterized by a molecular weight in the range 45000 to 60000 Dalton and wherein the molecular weight of iron sucrose is substantially unaltered when the composition is filled in a suitable container and is subjected to steam sterilization wherein, the composition is prepared by a process comprising the steps:
a) gradually adding an aqueous sodium carbonate solution over a period of 20 to 80 minutes to an aqueous ferric chloride solution at temperature of 10° C. to 30° C. wherein, quantity of sodium carbonate is 1.2 to 1.8 moles per mole of ferric chloride and isolating ferric oxyhydroxide cake thus formed,
b) suspending the ferric oxyhydroxide cake in water and adding sucrose to obtain a reaction mass wherein quantity of sucrose is 4 to 8 moles per mole of ferric chloride in step a,
c) adding sodium hydroxide to the reaction mass to obtain a pH of 10-12 and heating it to temperature of 100° C. to 106° C. for 12-36 hours,
d) adding methanol and filtering the reaction mass to obtain wet cake
e) preparing sodium sucrosate solution by dissolving sucrose and sodium hydroxide in a parenteral aqueous vehicle and heating at a temperature of 99° C. to 103° C. and
f) dissolving the wet cake in sodium sucrosate solution and removing methanol from the solution.
   and further wherein the volume is adjusted with the parenteral aqueous vehicle and the composition filled into suitable containers and subjected to steam sterilization.

The process involves gradually adding an aqueous sodium carbonate solution over a period of 20 to 80 minutes to an aqueous ferric chloride solution at temperature of 10° C. to 30° C., such as 11, 12, 13, 14, 1,5 16, 17, 18, 19, 20, 21, 22, 23, 24, 25, 26, 27, 28 or 29° C., within wherein, quantity of sodium carbonate is 1.2 to 1.8 moles per mole of ferric chloride, such as 1.3, 1.4, 1.5, 1.6 or 1.7 moles per mole of ferric chloride, and isolating ferric oxyhydroxide cake thus formed. The inventors have found that the temperature and rate of addition of sodium carbonate solution into ferric chloride solution are critical parameters in obtaining the iron sucrose of the desired characteristics. The addition time is dependent upon the temperature at which the addition is done. Thus, irrespective of the batch size, when the reaction is carried out at temperature of 10° C. to 30° C. the addition should be completed within 20 to 80 minutes, such as within about 25, 30, 35, 40, 45, 50, 55, 60, 65, 70 or 75 minutes. The preferred temperature during the addition is 15° C. to 19° C. and at this temperature the addition should be completed within 50 to 60 minutes. In, another embodiment, the addition can be performed at temperature of 13° C. to 16° C. Preferably, the quantity of sodium carbonate is 1.3 to 1.6 moles per mole of ferric chloride.

In one embodiment the process involves adding an aqueous sodium carbonate solution to an aqueous ferric chloride solution at temperature of 10° C. to 30° C. In one embodiment the temperature is about 15° C. to 19° C. In one embodiment the temperature is about 13° C. to 16° C.

In one embodiment the aqueous sodium carbonate solution is added to an aqueous ferric chloride solution within 20-80 minutes. In one embodiment the aqueous sodium carbonate solution is added to an aqueous ferric chloride solution within 50-60 minutes.

In one embodiment the quantity of sodium carbonate is 1.2 to 1.8 moles per mole of ferric chloride. In one embodiment the quantity of sodium carbonate is 1.3 to 1.6 moles per mole of ferric chloride.

Ferric oxyhydroxide thus formed can be isolated by appropriate techniques well known in the art for instance by filtration or decantation and the ferric oxyhydroxide cake is suspended in water and sucrose is added. The quantity of sucrose used is 4 to 8 moles per mole of ferric chloride used at the start of the process; preferably the quantity of sucrose is 5.5 to 6.5 moles per mole of ferric chloride. The pH of the reaction mass is then raised to 10-12 by addition of sodium hydroxide, preferably in the form of an aqueous solution. The reaction mass is then heated at 100° C. to 106° C. for 12 to 36 hours. The temperature and the time of heating are crucial parameters in controlling the molecular weight of the final iron sucrose. Preferably, the reaction mass is heated at 103° C. to 105° C. for 22 to 26 hours. More preferably, the reaction mass is heated at 103° C. to 105° C. for 23 to 24 hours. The reaction mass is then cooled and methanol is added into it. The quantity of methanol added should be sufficient to cause complete precipitation of the iron sucrose formed. The precipitated iron sucrose can be isolated by appropriate technique well known in the art for instance filtration or decantation. The precipitated iron sucrose is dissolved in sodium sucrosate solution. Sodium sucrosate solution is prepared by heating a mixture of sucrose, sodium hydroxide and water at about 99° C. to about 103° C. for about 22 to 26 hours. The reaction mass is concentrated to remove excess methanol from the mixture. The remaining solution may be filtered through a 0.2 micron filter and is filled in vials of desired size, sealed and subjected to steam sterilization. The conditions of steam sterilization are described under definition section of the specification.

In one embodiment the quantity of sucrose used is 4 to 8 moles per mole of ferric chloride used at the start of the process, such as 5, 6 or 7 moles per mole. For example, 5.5 to 6.5 moles per mole of ferric chloride.

In one embodiment the pH of the solution is raised to 10-12, such as 11.

In one embodiment the reaction mass is heated at 100° C. to 106° C., such as 101, 102, 103, 104 or 105° C. In one embodiment the reaction mass is heated to 103° C. to 105° C.

In one embodiment the reaction mass is heated for 12 to 36 hours, such as 13, 14, 15, 16, 17, 18, 19, 20, 21, 22, 23, 24, 25, 26, 2, 28, 29, 30, 31, 32, 33, 34, or 35 hours. For example 22 to 26 hours, or 23 to 24 hours.

In one embodiment sufficient methanol is added to cause complete precipitation of the iron sucrose formed.

In one embodiment precipitated iron sucrose if isolated using filtration or decantation.

In one embodiment the precipitated iron sucrose is dissolved in sodium sucrosate solution.

In one embodiment sodium sucrosate is prepared by heating sucrose, sodium hydroxide and water at about 99° C., such as about 98 to 100° C. In one embodiment the sodium sucrosate is prepared by heating sucrose, sodium hydroxide and water to about 103° C., such as 102 to 104° C. In one embodiment the sodium sucrosate is prepared by heating sucrose, sodium hydroxide and water for about 22 to 26 hours, such as 23, 24 or 25 hours.

In one embodiment the reaction mass is concentrated to remove excess methanol.

In one embodiment the remaining solution is filtered through a 0.2 to 2 micron filter, such as a 0.2 micron filter.

In another embodiment, the reaction mass obtained after dissolving iron sucrose in sodium sucrosate solution may be filtered through 0.2-2 micron filter, such as 0.3, 0.4, 0.5, 0.6, 0.7, 0.8, 0.9, 1.0, 1.1, 1.2, 1.3, 1.4, 1.5, 1.6, 1.7, 1.8 or 1.9 micron filter, and then concentrated to remove excess of methanol from the mixture. The volume of remaining solution may be adjusted with water for injection to adjust the iron content to 20±1 mg/mL and the solution is filled in vials of desired size, sealed and subjected to steam sterilization.

The present inventors have surprisingly found that the present process consistently yields iron sucrose having a molecular weight ranging from 45000 Dalton to 60000 Dalton. Moreover, the molecular weight of the iron sucrose prepared by the process of the present invention is not substantially altered when the composition is subjected to steam sterilization. In comparison, when the inventors tested the samples of the marketed iron sucrose Venofer®, marketed by Luitpold, the molecular weight of the sample increased when subjected to steam sterilization (Table-1).

TABLE 1
pH and Molecular weight data of Innovator
(VENOFER ®) formulation

| | Molecular Weight | | pH | |
|---|---|---|---|---|
| Venofer ® | | Autoclaved at | | Autoclaved at |
| Batch | Initial | 121° C./15 mins | Initial | 121° C./15 mins |
| 258101 | 52980 | 64510 | 10.72 | 10.37 |
| 317101 | 53055 | 63325 | 10.74 | 10.38 |

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The present invention is further illustrated in detail with reference to the following examples. It is desired that the examples be considered in all respect as illustrative and are not intended to limit the scope of the claimed invention.

### EXAMPLES

### Example-1

Ferric Chloride hexahydrate was dissolved at 15-25° C. under stirring (RPM 170-200 in Water for injection (WFI) and the solution was filtered through 2 micron filter. The content of above reactor was cooled to 15-20° C. under stirring (RPM 70-90). A 30% w/v sodium carbonate solution (cooled to 25-30° C.) was charged to the Ferric Chloride solution at 15-19° C. during 55±5 min using peristaltic pump through 40 micron filter. The mixture was stirred for 20 min at same temp (15-20° C.) at RPM 70-90. The slurry of ferric oxyhydroxide was filtered through 2 micron filter and the cake was washed with cold WFI (8-12° C.). The wet cake obtained was charged in another reactor followed by addition of cold WFI (8-12° C.) under stirring (RPM 70-90) under nitrogen. Sucrose was charged to the above mixture under stirring (RPM 70-90). A sodium hydroxide (30% w/v) aqueous solution was charged to the reaction mass through 2 micron filter. The reaction mass was heated to reflux temperature (100-106° C.) and maintained at reflux for 22-26 hour under Nitrogen and stirring (RPM 70-90). The reaction mass was cooled to 44-48° C. under stirring (RPM 70-90). Methanol was then charged into the reaction mass under stirring (RPM 70-90) through 2 micron filter. The reaction mass was stirred (RPM 15-25) for 5 hour and then allowed to settle (for not less than 7 hour). Supernatant was decanted using peristaltic pump. The settled mass was filtered through 1 micron filter. The reactor was washed with water:methanol (1:2). The wet cake was washed with water:methanol (1:2). The wet cake obtained after filtration was dissolved in sodium sucrosate solution (prepared by heating a mixture of WFI, Sucrose and NaOH to 99-103° C. for 22-26 hour under reflux condition under stirring at RPM 70-90) with stirring (RPM 10-20). The solution was filtered through 1 micron filter and remaining sodium sucrosate solution was charged into the reactor through 1 micron filter cloth. The reaction mass was then concentrated at 30-60° C. under vacuum and stirring (RPM 40-60) to remove methanol.

The solution was filled in the vials of desired volume and was sterilized in an autoclave with following conditions—Temperature 121° C., Time: 15 minutes, Pressure: about 15 psi.

### Example-2

Ferric Chloride hexahydrate was dissolved at 15-25° C. under stirring (RPM 170-200 in Water for injection (WFI) and the solution was filtered through 1.2 micron filter. The content of above reactor was cooled to 13-17° C. under stirring (RPM 70-90). A 30% w/v sodium carbonate solution (cooled to 25-30° C.) was charged to the Ferric Chloride solution at 13-16° C. during 55±5 min using peristaltic pump through 40 micron filter. The mixture was stirred for 20 min at same temp (13-16° C.) at RPM 70-90. The slurry of ferric oxyhydroxide was filtered through 2 micron filter cloth and the cake was washed with cold WFI (8-12° C.). The wet cake obtained was charged in another reactor followed by addition of cold WFI (8-12° C.) under stirring (RPM 70-90) under nitrogen. Sucrose was charged to the above mixture under stirring (RPM 70-90). A sodium hydroxide (30% w/v) aqueous solution was charged to the reaction mass through 40 micron filter. The reaction mass was heated to reflux temperature (100-106° C.) and maintained at reflux for 22-26 hour under nitrogen and stirring (RPM 70-90). The reaction mass was cooled to 44-48° C. under stirring (RPM 70-90). Methanol was then charged into the reaction mass under stirring (RPM 70-90) through 1.2 micron filter. The reaction mass was stirred (RPM 15-25) for 5 hour and then allowed to settle (for not less than 7 hour). Supernatant was decanted from using peristaltic pump. The settled mass was filtered through 1 micron filter cloth. The reactor was washed with water:methanol (1:2). The wet cake was washed with water:methanol (1:2). The wet cake obtained after filtration was dissolved in sodium sucrosate solution (prepared by heating a mixture of WFI, Sucrose and NaOH to 99-103° C. for 22-26 hour under reflux condition under stirring at RPM 70-90) with stirring (RPM 10-20). The solution was filtered through 1 micron filter cloth. The reaction mass was then concentrated at 30-60° C. under vacuum and stirring (RPM 40-60) to remove methanol and diluted with WFI to adjust iron content 20±1 mg/mL.

The solution was filled in the vials of desired volume and was sterilized in an autoclave with following conditions—Temperature 121° C., Time: 15 minutes, Pressure: about 15 psi.

### Example-3

Ferric Chloride hexahydrate was dissolved at 15-25° C. under stirring (RPM 170-200 in Water for injection (WFI) and the solution was filtered through 1.2 micron filter. The content of above reactor was cooled to 21-23° C. under stirring (RPM 70-90). A 30% w/v sodium carbonate solution (cooled to 25-30° C.) was charged to the Ferric Chloride solution at 21-23° C. during 30±5 min using peristaltic pump through 40 micron filter. The mixture was stirred for 20 min at same temp (13-16° C.) at RPM 70-90. The slurry of ferric oxyhydroxide was filtered through 2 micron filter cloth and the cake was washed with cold WFI (8-12° C.). The wet cake obtained was charged in another reactor followed by addition of cold WFI (8-12° C.) under stirring (RPM 70-90) under nitrogen. Sucrose was charged to the above mixture under stirring (RPM 70-90). A sodium hydroxide (30% w/v) aqueous solution was charged to the reaction mass through 40 micron filter. The reaction mass was heated to reflux temperature (100-106° C.) and maintained at reflux for 22-26 hour under nitrogen and stirring (RPM 70-90). The reaction mass was cooled to 44-48° C. under stirring (RPM 70-90). Methanol was then charged into the reaction mass under stirring (RPM 70-90) through 1.2 micron filter. The reaction mass was stirred (RPM 15-25) for 5 hour and then allowed to settle (for not less than 7 hour). Supernatant was decanted from using peristaltic pump. The settled mass was filtered through 1 micron filter cloth. The reactor was washed with water:methanol (1:2). The wet cake was washed with water:methanol (1:2). The wet cake obtained after filtration was dissolved in sodium sucrosate solution (prepared by heating a mixture of WFI, Sucrose and NaOH to 99-103° C. for 22-26 hour under reflux condition under stirring at RPM 70-90) with stirring (RPM 10-20). The solution was filtered through 1 micron filter cloth. The reaction mass was then concentrated at 30-60° C. under vacuum and stirring (RPM 40-60) to remove methanol and diluted with WFI to adjust iron content 20±1 mg/mL.

Table-2 provides the Molecular weight and pH of the composition prepared by the process of present invention before and after autoclave (at 121° C./15 min).

TABLE 2
pH and Molecular weight composition of Example 1

| | Molecular Weight | | | pH | | |
|---|---|---|---|---|---|---|
| | | 1^{st} | 2^{nd} | | 1^{st} | 2^{nd} |
| | | Auto- | Auto- | | Auto- | Auto- |
| Batch | Initial | clave | clave | Initial | clave | clave |
| HKMP0900 | 52321 | 52628 | 52498 | 11 | 10.82 | 10.65 |
| HKMP0939 | 59041 | 59113 | 58748 | 10.98 | 10.85 | 10.75 |

1. A composition comprising iron sucrose dissolved in aqueous vehicle suitable for parenteral administration wherein the iron sucrose is characterized by a molecular weight in the range 45000 to 60000 Dalton and wherein the molecular weight of iron sucrose is not substantially altered when the composition is filled in a suitable container and is subjected to steam sterilization.

2. The composition as claimed in claim 1 wherein, the iron sucrose in the composition is characterized by molecular weight in the range 50000 to 60000 Dalton.

3. A process for preparation of iron sucrose comprising:
a) gradually adding an aqueous sodium carbonate solution over a period of 20 to 80 minutes to an aqueous ferric chloride solution at temperature of 10° C. to 30° C. wherein, quantity of sodium carbonate is 1.2 to 1.8 moles per mole of ferric chloride and isolating ferric oxyhydroxide cake thus formed,
b) suspending the ferric oxyhydroxide cake in water and adding sucrose to obtain a reaction mass wherein quantity of sucrose is 4 to 8 moles per mole of ferric chloride in step a,
c) adding sodium hydroxide to the reaction mass to obtain a pH of 10-12 and heating it to temperature of 100° C. to 106° C. for 12-36 hours,
d) adding methanol and filtering the reaction mass to obtain wet cake
e) preparing sodium sucrosate solution by dissolving sucrose and sodium hydroxide in a parenteral aqueous vehicle and heating at a temperature of 99° C. to 103° C. and
f) dissolving the wet cake in sodium sucrosate solution and removing methanol from the solution.

4. The process for preparation of iron sucrose as in claim 3 wherein in step a, the aqueous sodium carbonate solution is added over a period of 50 to 60 minutes to the aqueous ferric chloride solution at temperature of 15° C. to 19° C. wherein, the quantity of sodium carbonate is 1.3 to 1.6 moles per mole of ferric chloride; in step b the quantity of sucrose is 5 to 6 moles per mole of ferric chloride and in step c the reaction mass is heated to temperature of 103° C. to 106° C. for 22 to 26 hours.

5. The process for preparation of iron sucrose as in claim 4 wherein in step a, the aqueous sodium carbonate solution is added to the aqueous ferric chloride solution at temperature of 13° C. to 16° C.

6. The composition of claim 1 for use in the treatment of anemia.
